(19) United States
(12) Patent Application Publication
(10) Pub. No.: US 2006/0089570 A1
(43) Pub. Date: Apr. 27, 2006

(54) Methods and devices for cervix measurement

(76) Inventors: Hebah Noshy Mansour, La Mirada, CA (US); Ramez Emile Necola Shehada, La Mirada, CA (US)

Correspondence Address: HEBAH NOSHY MANSOUR, 14759 HARDAWAY DR., LA MIRADA, CA, 90638, US

(21) Appl. No.: 11/259,513

(22) Filed: Oct. 25, 2005

Related U.S. Application Data
(60) Provisional application No. 60/621,726, filed on Oct. 25, 2004.

(51) Int. Cl. A61B 5/103; A61B 8/00

(52) U.S. Cl. 600/591; 600/437

(57) ABSTRACT

The present invention is directed to a system and method for measuring the dilatation and effacement of the uterine cervix in a manner that is non-invasive to the cervix. The system having a probe and a monitoring unit serve to measure the cervix dimensions during routine clinical visits and is also suitable for personal checkup at home. The probe primarily includes a camera for imaging the cervix and a set of circles of different diameters imprinted on the imaging window of the probe. The probe is inserted into the vagina until its imaging window abuts the cervix. The system captures and displays images of the cervix opening superimposed with the set of concentric circles, which allows the user to perform a visual comparison and determine the diameter of the opening. The probe may also include an ultrasonic transducer operating in pulse-echo mode to measure the thickness of the cervix and determine its effacement.

### CROSS REFERENCE TO RELATED APPLICATIONS

This application claims the benefit of U.S. Provisional Application No. 60/621,726, filed Oct. 25, 2004, entitled Cervix Monitor, the entire contents of which application are incorporated herein by reference.

### FIELD OF THE INVENTION

The present invention relates to a method and system for measuring the dimensions of the uterine cervix, in particular, the size of the opening of the cervix and the thickness of the cervix.

### BACKGROUND OF THE INVENTION

Measuring the cervix dimensions, in particular the diameter of the opening of the cervix and the thickness of the cervix is desirable during pregnancy, in particular the late stages of pregnancy, because the dimensions of cervix can be an indicator of the woman's susceptibility to preterm labor (or premature labor, used interchangeably herein). In current clinical practice, the diameter of the opening of the cervix (or cervical diameter, used interchangeably herein) and the thickness of the cervix (or cervical effacement, used interchangeably herein) is performed manually by inserting a gloved hand into the vagina and then using the fingers to probe the diameter and depth of the opening of the cervix. This method is known as digital probing and suffers several inherent limitations, including the following. First, the method is approximate as the accuracy of the measurement depends on the experience of the health care provider. Second, the method can cause discomfort to the patient during each session of digital probing which may be performed repeatedly. Third, hand examinations can increase the risk of infection to the to the mother and the fetus despite the use of gloves. Fourth, the method is known to induce labor and therefore should be avoided especially in women susceptible to preterm labor or suffering from an incompetent cervix.

There have been many attempts to develop devices for accurate and user-independent measurement of the cervical diameter and effacement. However, previous techniques failed to gain wide clinical acceptance due to several limitations, including the complexity of use, inaccuracy of measurements, tissue trauma caused by the devices or their components, including the manner by which the components are attached to the cervix, costly sterilization between uses, and/or patient discomfort.

Consequently, the manual method of digital probing continues to be a favored method of monitoring cervical diameter and effacement. Therefore, there exists a desire for a system and method to measure the cervical diameter and effacement in a manner that is noninvasive to the cervix and preferably that is minimally invasive to the patient.

### SUMMARY OF THE INVENTION

The present invention is directed to a system and method for measuring the dilatation and effacement of the uterine cervix in a manner that is non-invasive to the cervix.

The system having a probe and a monitoring unit serve to measure the cervix dimensions during routine clinical visits and is also suitable for personal checkup at home. The probe primarily includes a camera for imaging the cervix, a lens to provide an optimal field of view for the camera at close range to the cervix, a light source to illuminate the cervix, and a set of circles corresponding to different diameters imprinted on the imaging window of the probe. Advantageously, the probe is configured to provide a predetermined distance from which the camera has an appropriate range to image the cervix opening. The probe is inserted into the vagina until the imaging window of the probe abuts the cervix. The system captures and displays images of the cervix opening superimposed with the set of concentric circles, which allows the user to perform a size comparison between the opening and the circles and determine the diameter of the opening. The probe may also include an ultrasonic transducer operating in pulse-echo mode to measure the thickness of the cervix and determine its effacement. The images provided by the camera facilitate the positioning of the ultrasound transducer on the lip of cervix in a minimally invasive manner.

A handle portion of the probe facilitates the insertion and removal of the probe from the vagina and protects electrical connections between the probe and the monitoring unit. The handle portion is generally rigid or preferably semi flexible but is adapted to have sufficient rigidity to facilitate the insertion of the probe into the vagina.

### DETAILED DESCRIPTION

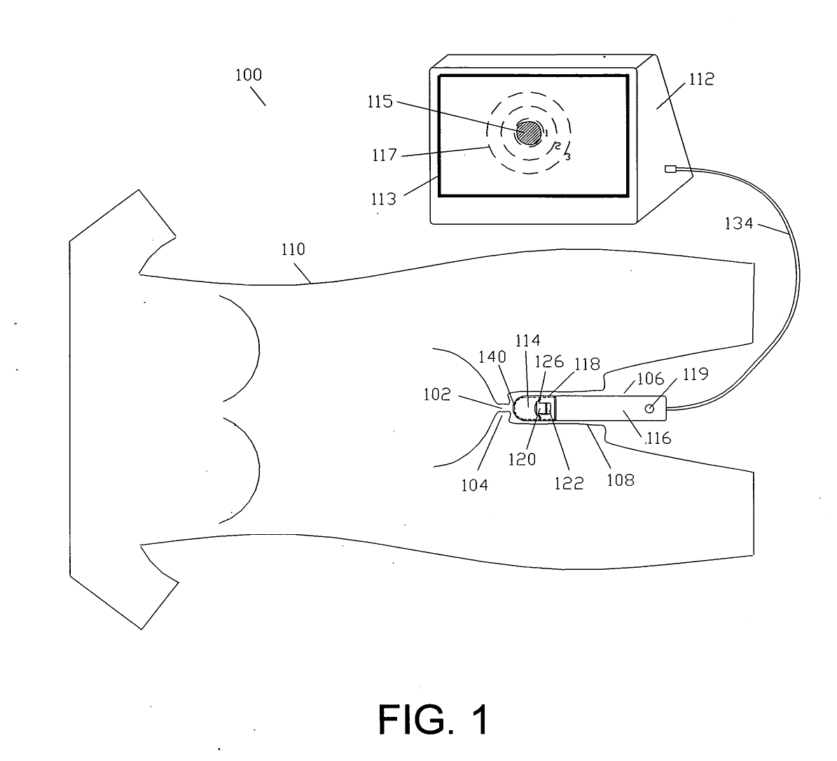

Referring to FIG. 1, the present invention includes a system 100 for measuring the diameter of the opening 102 of the cervix 104 in a manner that is non-invasive to the cervix. The system includes a probe 106 that is inserted into a vagina 108 of the patient 110 to gather data relating to the cervix 104, in particular, the diameter of the opening 102, and a monitoring unit 112 that is in communication with the probe 106 and receives image data from the probe 106 which is displayed to a health care provider located proximately to the patient. Accordingly, the patient need not have her cervix digitally probed.

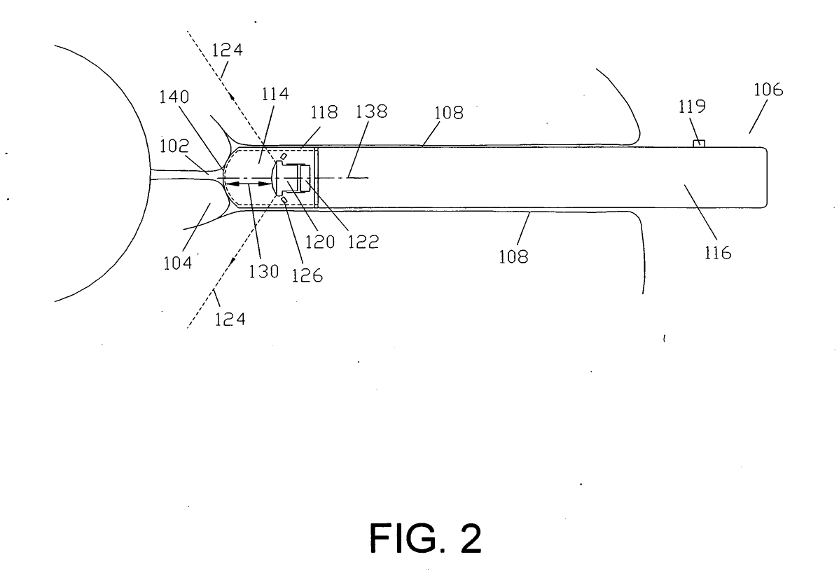

Referring to FIGS. 1 and 2, a preferred embodiment of the cervix probe 106 includes generally a distal probe head 114 and a proximal probe handle 116 extending proximally from the probe head. The probe head 114 includes a housing 118 encapsulating a lens 120 and a camera 122 that is proximal of the lens and adapted to capture image data of objects within a predetermined field of view 124 of the lens. The housing 118 also contains at least one light source 126 positioned to illuminate the field of view 124 of the lens inside the vagina. The lens 120, the camera 122 and the light source 126 are fixedly mounted within the housing 118.

The housing 118 may be generally cylindrical in shape along a longitudinal axis 138 with preferably a streamlined distal end 140 to facilitate insertion into the vagina 108. The housing has a length in the range of about 4 cm to 7 cm, and more preferably about 5 cm. The housing has a diameter of about 3 cm. The housing 118 may preferably be made of an optically transparent plastic material such as, for example, polycarbonate or acrylic; however, other materials such as Pyrex may be used. The housing 118 may be hermetic to protect its internal components from contamination by the outside environment. The housing 118 may also be coated or made of a hydrophobic (i.e. water repellent) material to prevent fluids, if any, from adhering to its exposed surfaces.

The camera 122 is of the miniature type with a size ranging between about 16×16×8 mm and 6×6×3 mm and more preferably 8×8×4 mm. The lens 120 may be a wide angle lens, its field of view (FOV) 124 ranging between about 120 to 190 degrees and more preferably about 170 to 180 degrees to enable imaging of the cervix 104 from a relatively close range. In that regard, the range is generally provided by a predetermined distance or separation 130 between the lens 120 and the distal end 140 of the housing 118 which is also generally the distance between the lens 120 and the cervix 104, since the probe 106, as described below in further detail, is inserted into the vagina with the distal end 140 generally abutting the cervix 104 or the cervix opening 102. Accordingly, the camera 122 and the lens 120 are selectively and fixedly situated within the housing 118 at the distance 130 from the distal end 140 of the housing 118. In particular, the distance 130 is selected to provide enough range to allow the field of view 124 of the lens 122 to capture a dilated cervix opening (at about 4 cm or so) when the distal end 140 is abutting, or is proximate to, the cervix 104 or the opening 102. The distance 130 may range between about 1 cm to 2.5 cm, and more preferably about 2 cm. The camera 122 may be of any type, including color, grayscale, CCD, CMOS, analog, digital, multispectral, or thermal. Optical filters may also be used to remove certain wavelength bands to enhance the image and/or clarify features of interest such as the opening 102 of the cervix 104.

One or more light sources 126 are disposed around the camera 122 to provide the proper illumination necessary for imaging without saturating the camera 122 by, for example, internal reflection. The light sources 126 may be light emitting diodes (LEDs) of the miniature surface mount type (SMD), bulbs, or optical fibers. The optical fibers, if used, may have a tip that is polished at an angle to provide side emission. The light sources 126 may emit white or monochromatic light at certain wavelengths, including infrared, to provide better viewing of different tissues/materials, glare reduction and/or improved imaging. The light sources 126 may be aimed at different angles and may be illuminated simultaneously, individually and/or in groups to improve imaging and/or image quality and avoid saturation of the camera 122.

The probe handle 116 is rigid or preferably semi flexible to enable the insertion of the probe into the vagina 108. The outer diameter of the handle 116 is no greater and preferably smaller than the diameter of the housing 118 as shown in FIGS. 1 and 2. The handle 116 is preferably long enough to allow it to exit the vagina 108 when the distal end 140 of the housing 118 is abutting the cervix 104. A suitable length ranges about 14 cm to 25 cm and more preferably about 16 cm to 20 cm. The probe handle 116 may be continuous with the probe head 114 and made of the same material such as polycarbonate or acrylic plastic. Alternatively, the handle 116 may be made of a semi flexible material such as silicone, Tygon or any other semi flexible rubber. The probe handle 116 and may be hollow to accommodate a battery pack (not shown) and allow the passage of the cable 134 that includes the electrical wires to and from the camera 122. The probe handle 116 may be configured with an aperture to enable the cable 134 to exit at or near the proximal end of the handle and extend to the monitoring unit 112. It is understood by one of ordinary skill in the art that the electrical connection between the probe 106 and the monitoring unit 112 by which image data and/or control signals are sent and received need not be accomplished by wires but that it can be wireless as well, or a combination of the two.

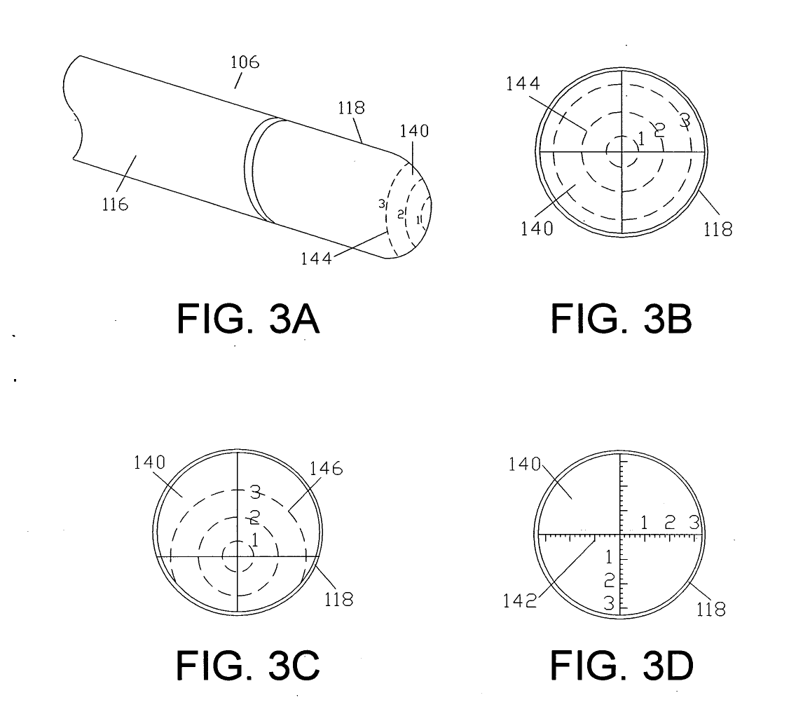

The distal end 140 of the housing 118 has a set of concentric circles 144 having different diameters imprinted on the transparent housing 118 as shown in FIGS. 3A and 3B. Each circle is tagged with a number that indicates the diameter that it represents when imaged by the camera 122. For example the diameter of the innermost circle may correspond to 1-cm, the next circle may correspond to 2-cm, and so forth. The camera 122 is configured to image the set of concentric circles 144 superimposed over the image of the cervix 104 that abuts the distal end 140 when the probe 106 is properly applied. The actual diameter of each circle may be adjusted to compensate for the variable radial magnification of the wide-angle lens 120 and/or the optical distortion caused by the curvature of the distal end 140. Accordingly, the actual diameter of each circle may be different from the physical diameter that it represents. The circles may be continuous, dashed or dotted, and may be color-coded to facilitate their visual identification. The set of concentric circles 144 may be printed with a fluorescent or a semi-reflective material that would glow when illuminated by the light source 126 and appear to the camera 122 as haloes to improve their visibility.

Alternatively, the set of concentric circles 146 may be off-centered from the longitudinal axis 138 of the probe head 114 as shown in FIG. 3C to accommodate for the angulation of the cervix 104 relative to the vagina 108. This off-centricity may improve the overlap of the circles 146 with the opening 103 of the cervix and therefore simplify the visual comparison to identify the circle that best matches the size of the opening 102.

Alternatively, a graduated crosshairs 142 may be imprinted on the transparent housing 118 as shown in FIG. 3D.

Yet alternatively, the set of concentric circles may be electronically generated and superimposed on the image of the camera 122. The user may select to turn off the electronically generated circles when positioning the probe next to the cervix and turn them on when ready to take the measurements.

The monitoring unit 112 has a screen 113 that displays the image 115 of the opening 102 superimposed with images 117 of the circles 144 (or the off-centered circles 146, or the graduated crosshairs 142). For this probe embodiment, the monitoring unit 112 may be a video monitor, a TV, or a computer.

In a typical probe application procedure, the probe 106 is inserted into the vagina 108 until the distal end 140 of the housing 118 touches the cervix 104. This may be done under live video guidance from the camera 122 or by determining that further insertion of the probe is blocked by the cervix 104. The camera 122 images the cervix 104 with the superimposed set of concentric circles 144 (or the off-centered circles 146, or the graduated crosshairs 142) and transmits the images to the monitoring unit 112. The orientation of the probe 106 may be adjusted such that the circles 144 (or the off-centered circles 146, or the graduated crosshairs 142) coincide with the cervix opening 102. The diameter of the cervix opening 102 may be determined by identifying the circle that best matches the size of opening 102.

It is understood by one of ordinary skill in the art that the electrical connection between the probe 106 and the monitoring unit 112 by which image data and/or control signals are sent and received need not be accomplished by wires but that it can be wireless as well, or a combination of the two. The cervix probe 106 may include a wireless transmitter (not shown) that can transmit real-time images of the cervix directly to a local TV tuned to a predetermined channel, or to a dedicated video receiver that is connected to a TV or a video monitor.

In an alternative embodiment, the probe may include a processor (not shown) to superimpose an electronically generated dynamic circle (not shown) over the images of the cervix 104 captured by the camera 120. The diameter and the position of the dynamic circle may be varied using control buttons 119 located on the handle 116 to achieve a visual overlap and size match with the underlying image 115 of the opening 102. A numerical indicator (not shown) on the screen 113 may be used to indicate the actual physical diameter corresponding to the dynamic circle. The effective diameter of the dynamic circle may vary depending on its location within the field of view 124 of the wide-angle lens 120 and proper calibration should be applied.

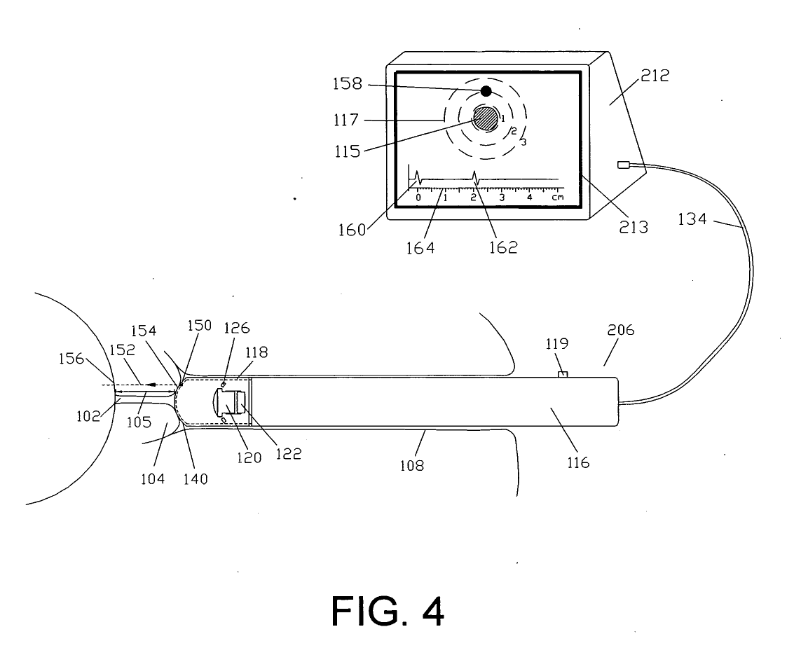

Another embodiment of the system 100 shown in FIG. 4 includes an ultrasound transducer 150 to measure the thickness 105 of the cervix 104. Other than the addition of the ultrasound transducer 150, the probe 206 is similar to the probe 106 in all components, functions and applications.

The ultrasound transducer 150 is positioned in the probe head 114, and preferably positioned in the distal end 140, to contact the cervix 104 when the probe 206 is fully inserted into the vagina 108. The transducer 150 lies within the field of view 124 of the lens 120 and appears as a small spot 158 on the image of the cervix 104 captured by the camera 122 and displayed on the screen 213 of the monitoring unit 212. Live video images from the camera 122 may be used to orient the probe 206 and position the small spot 158 representing the transducer 150 on the rim (or lip) of the cervix 104. The back of the ultrasound transducer 150 may be coated with a fluorescent or a semi-reflective material to enhance its visualization within the field of view 124. The ultrasound transducer 150 may be a circular piezoelectric element with a diameter between 1 to 5 mm, and more preferably between 2 to 3 mm. The transducer is driven using a micro-coaxial cable (e.g. AWG-40) to maximize visibility around the transducer 150.

The ultrasound transducer 150 may be operated in A-mode (Amplitude mode) in a similar fashion to common corneal pachemetry. In a typical A-mode operation, the ultrasound transducer 150 transmits ultrasound pulses into the cervix 104 and receives echoes returned from the acoustical interfaces along the travel path 152. A first major echo 160 may be received from the transducer/cervix interface 154 and a second major echo 162 may be received from the cervix/uterus acoustical interface 156. The arrival time of the first echo 160 and the second echo 162 may be converted to a distance scale 164 using the known velocity of sound in the tissue (about 1500 m/s) and displayed on the screen 213 of the monitoring unit 212. The distance difference between the first echo 160 and the second echo 162 represents the thickness 105 of the cervix 104 and may be read directly from the distance scale 164.

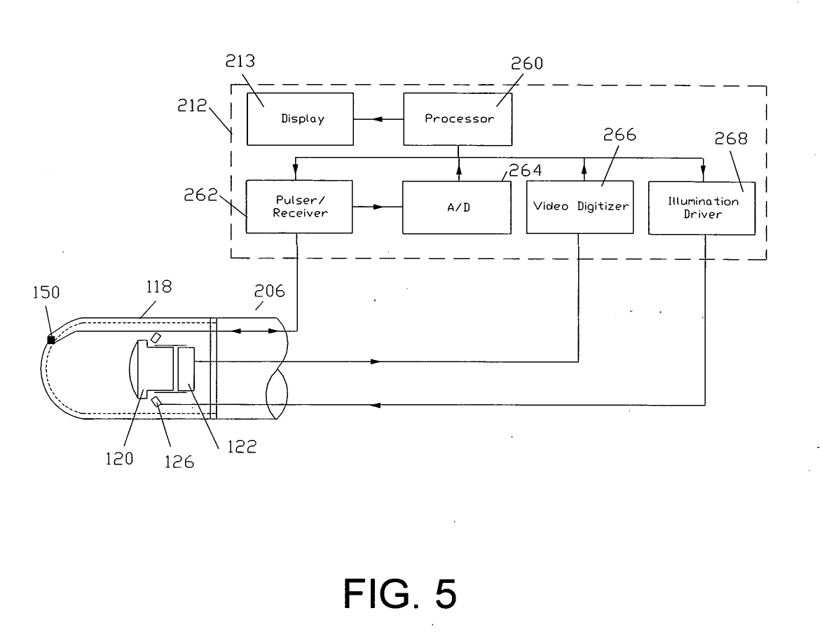

A block diagram showing the main components of the monitoring unit 212 controlling the probe 206 is shown in FIG. 5. The monitoring unit 212 includes a processor 260, a display 213, an ultrasonic pulser/receiver 262, an analog to digital converter 264, a video digitizer 266, and an illumination driver 268. In a typical operation, the processor 260 triggers the ultrasonic pulser/receiver 262 to pulse the transducer 150 within the probe head 114 to transmit an ultrasonic pulse into the cervix 104 as shown in FIG. 4. The ultrasound echoes returned from the cervix 104 are amplified by the ultrasonic pulser/receiver 262 and digitized by the analog to digital converter 264. The processor 260 processes the digitized echoes to determine the time between the first echo 160 and the second echo 162 and convert the time scale to a distance scale 164 using the known velocity of sound in tissue (about 1500 m/s). The processor 260 determines the thickness 105 of the cervix 104 by calculating the distance difference between the first echo 160 and the second echo 162. The echoes 160 and 162 may also be displayed along a distance scale 164 on the screen 213.

Simultaneously, the illumination driver 268 powers up the light sources 126 to illuminate the field of view 124 and enable imaging of the cervix 104 by the camera 122. The images acquired by the camera 122 are digitized by the video digitizer 266 and processed by the processor 260 to correct for the spatial distortions caused by the wide-angle lens and/or the curvature of the distal end 140.

The captured image of the cervix may be preprocessed to correct for any spatial distortion caused by the wide-angle lens 120 of the camera 122. The wide-angle lens 120 (i.e. fisheye) may be used to enable the imaging of the cervix 104 from a relatively close distance of approx. 2 cm. Wide-angle lenses can introduce an image distortion known as the barrel distortion, which is caused by the uneven magnification between the edges and the center of the lens. Barrel distortion is a type of radial distortion in which horizontal and vertical lines appear to be bent outwards toward the edges of the image. Algorithms to correct barrel distortion in images are readily available in the literature, e.g., Mundhenk, T. N., et al., “Techniques for fisheye lens calibration using a minimal number of measurements,” Proceedings of the SPIE, SPIE-Int. Soc. Opt. Eng., 4197, pp. 181-90, 2000, and e.g. James P. Helferty, et al., “Videoendoscopic Distortion Correction and Its Application to Virtual Guidance of Endoscopy,” IEEE Transactions on Medical Imaging, Vol. 20, No. 7, pp 605-617, 2001. These algorithms may be applied to the images captured by the video digitizer (not shown) to minimize or remove barrel distortion. In addition, algorithms for the correction of perspective distortion (e.g. Waltz, F. M., “Implementation of real-time perspective correction,” Proceedings of the SPIE—SPIE-Int. Soc. Opt. Eng. 849, pp. 179-83, 1988) may be also applied to correct for distortions caused by the non-perpendicular imaging of the cervix (i.e. when the probe is at a tilted viewing angle of the cervix). The distortion-corrected images may be color balanced and filtered using, for example, a median filter to improve image quality.

As shown in FIG. 4, the screen 213 displays an image 115 of the cervix opening 102 superimposed with the images 117 of the set of concentric circles 144 for a size match to determine the diameter of the opening 102. Simultaneously, the screen 213 displays the echoes 160 and 162 on the distance scale 164 to indicate the thickness 105 of the cervix 104.

Alternatively, The processor 260 may also generate a dynamic circle (not shown) that can be used to determine the diameter of the opening 102 of the cervix 104 as described in the above embodiments.

Although the above detailed description describes and illustrates various preferred embodiments, the invention is not so limited. Many modifications and variations will now occur to persons skilled in the art. As such, the preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims, which are to have their fullest and fair scope.

What is claimed is:

1. A system for measuring dilatation of a cervix opening, comprising:
a probe configured for placement in a vagina for imaging the cervix and generating image data;
a probe imaging window configured to abut the cervix when the probe is placed in the vagina;
a mark superimposed on the image data; and
a display unit for displaying the image data superimposed by the mark.

2. A system of claim 1, wherein said mark includes a set of circles having different diameters.

3. A system of claim 2, wherein each circle in said set of circles is tagged with a number indicating its corresponding diameter.

4. A system of claim 2, wherein each circle in said set of circles is color coded to indicate its corresponding diameter.

5. A system of claim 1, wherein said mark includes a graduated crosshairs.

6. A system of claim 1, wherein said mark is imprinted on said probe imaging window.

7. A system of claim 4, wherein said mark is imprinted with a fluorescent material.

8. A system of claim 1, wherein said mark is electronically generated.

9. A system of claim 1, wherein said mark is dynamic.

10. A system for measuring dimensions of a cervix, comprising:
a probe configured for placement in a vagina for imaging a cervix and generating image data;
a probe distal end configured to abut the said cervix when the said probe is placed in said vagina;
a mark superimposed on the image data;
an ultrasound transducer configured to probe said cervix and generate ultrasonic data; and
a processor configured to process the image data and the ultrasonic data to provide dimensions of the said cervix.

11. A system of claim 10, wherein said mark includes a set of circles having different diameters.

12. A system of claim 10, wherein said mark is electronically generated.

13. A system of claim 1, wherein said mark is dynamic.

14. A system of claim 10, wherein said mark includes a graduated crosshairs.

15. A system of claim 10, wherein said ultrasound transducer operates in pulse-echo mode.

16. A system of claim 10, wherein the image data is used to position said ultrasound transducer on the cervix.

17. A method for measuring a diameter of a cervix opening using a probe, a mark, and an image display, comprising:
the probe capturing an image of the cervix opening and providing image data;
the mark superimposed on the image data;
the image display displaying the mark superimposed on the image data; and
compare the said image data to the said mark to determine the said diameter of the said cervix opening.

18. A method of claim 17, wherein said mark includes a set of concentric circles.

19. A method of claim 17, wherein said mark includes a graduated crosshairs.

20. A system of claim 17, wherein said mark is dynamic.
